(19) United States
(12) Patent Application Publication
(10) Pub. No.: US 2023/0380504 A1
(43) Pub. Date: Nov. 30, 2023

(54) VAPORIZER AND ELECTRONIC VAPORIZATION DEVICE

(71) Applicant: Jiangmen Smoore New Material Co., Ltd., Jiangmen (CN)

(72) Inventor: Haoliang ZHU, Jiangmen (CN)

(21) Appl. No.: 18/308,359

(22) Filed: Apr. 27, 2023

(30) Foreign Application Priority Data
Apr. 29, 2022 (CN) 202221043804.6

(51) Int. Cl. A24F 40/485; A24F 40/42; A24F 40/10

(52) U.S. Cl. CPC A24F 40/485; A24F 40/42; A24F 40/10; A24F 40/46

(57) ABSTRACT

A vaporizer includes: a shell; a vaporization assembly having a sleeve and a vaporization core, the sleeve being arranged in the shell, a liquid storage cavity being defined between the shell and the sleeve, the vaporization core being arranged in the sleeve, and a liquid inlet hole communicating the vaporization core with the liquid storage cavity being provided on the sleeve; and a vent tube sleeved between the sleeve and the vaporization core, and spaced apart from at least one of the sleeve or the vaporization core to form a vent channel. The vent channel is arranged so as to be in communication with the liquid inlet hole and an outside.

### CROSS-REFERENCE TO PRIOR APPLICATION

Priority is claimed to Chinese Patent Application No. 202221043804.6, filed on Apr. 29, 2022, the entire disclosure of which is hereby incorporated by reference herein.

### FIELD

The utility model relates to the field of vaporization technologies, and in particular, to a vaporizer and an electronic vaporization device.

### BACKGROUND

An aerosol is a colloidal dispersion system formed by solid or liquid small particles dispersed and suspended in a gas medium. Because the aerosol may be absorbed by a human body through the respiratory system, a new alternative absorption method is provided for users. For example, a vaporization device that can bake and heat an herb or ointment aerosol-generation substrate to generate aerosols is applied to different fields to deliver inhalable aerosols to the users to replace conventional product forms and absorption methods.

An electronic vaporization device usually uses a vaporizer to heat and vaporize an aerosol-generation substrate. In the related art, a cotton core vaporization core is plugged in a sleeve, and when the aerosol-generation substrate in a liquid storage cavity flows continuously to the vaporization core through a liquid inlet hole on the sleeve, a negative pressure will be formed in the liquid storage cavity, which will cause obstructed liquid feeding of the aerosol-generation substrate.

### SUMMARY

In an embodiment, the present invention provides a vaporizer, comprising: a shell; a vaporization assembly comprising a sleeve and a vaporization core, the sleeve being arranged in the shell, a liquid storage cavity being defined between the shell and the sleeve, the vaporization core being arranged in the sleeve, and a liquid inlet hole communicating the vaporization core with the liquid storage cavity being provided on the sleeve; and a vent tube sleeved between the sleeve and the vaporization core, and spaced apart from at least one of the sleeve or the vaporization core to form a vent channel, wherein the vent channel is configured to be in communication with the liquid inlet hole and an outside.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic structural diagram of a vaporizer according to an embodiment of the utility model;

FIG. 2 is a schematic exploded view of a vaporizer according to another embodiment of the utility model; and

FIG. 3 is a schematic diagram of partial assembly of the vaporizer shown in FIG. 2.

### DETAILED DESCRIPTION

In an embodiment, the present invention provides a vaporizer and an electronic vaporization device for the problem of obstructed liquid feeding of the aerosol-generation substrate in the related art.

In an embodiment, the present invention provides a vaporizer. The vaporizer includes: a shell;
a vaporization assembly, including a sleeve and a vaporization core, where the sleeve is arranged in the shell, a liquid storage cavity is defined between the shell and the sleeve, the vaporization core is assembled in the sleeve, and a liquid inlet hole communicating the vaporization core with the liquid storage cavity is provided on the sleeve; and
a vent tube, where the vent tube is sleeved between the sleeve and the vaporization core, and spaced apart from at least one of the sleeve or the vaporization core to form a vent channel, where
the vent channel is capable of being in communication with the liquid inlet hole and the outside.

In the foregoing vaporizer, an aerosol-generation substrate in the liquid storage cavity can directly flow to the vaporization core through the liquid inlet hole, to supply liquid to the vaporization core. In addition, the vaporizer further includes a vent tube, where the vent tube is sleeved between the sleeve and the vaporization core, and spaced apart from at least one of the sleeve or the vaporization core to form a vent channel, where the vent channel is capable of being in communication with the liquid inlet hole and the outside. In this way, by arranging the vent tube between the vaporization core and the sleeve, a gap between the vent tube and at least one of the vaporization core or the sleeve forms the vent channel. When liquid feeding of the aerosol-generation substrate is from the liquid storage cavity and the liquid flows to the vaporization core, the external air can flow to the liquid inlet hole and the liquid storage cavity through the vent channel, to prevent formation of a negative pressure in the liquid storage cavity, and keep the air pressure in the liquid storage cavity in balance with the atmospheric pressure outside, thereby ensuring smooth liquid feeding.

In an embodiment, a gap of the vent channel ranges from 0.05 mm to 0.15 mm.

In an embodiment, the vaporization core includes a base body and a core body, the base body is sleeved in the sleeve, and the core body is at least partially mounted on the base body, where
a through hole communicating the liquid inlet hole with the core body is provided on the base body; and the vent tube is sleeved between the base body and the sleeve, and sleeved with at least one of the base body or the sleeve in a spaced-apart manner to form the vent channel.

In an embodiment, the vent tube is fixedly sleeved on the base body, and a gap is reserved between the vent tube and the sleeve in a radial direction and the vent channel is defined.

In an embodiment, the vent tube is fixedly sleeved on an inner wall of the sleeve, and a gap is reserved between the vent tube and the base body in a radial direction and the vent channel is defined.

In an embodiment, a gap is reserved between the vent tube and each of the sleeve and the base body in a radial direction and the vent channel is defined.

In an embodiment, a vent groove extending and provided in an axial direction of the vent tube is provided on the base body, one part of the vent groove is inside the vent tube, and the other part of the vent groove extends to be in communication with the through hole, and the vent groove is constructed to be the vent channel.

In an embodiment, the sleeve includes a first tube section and a second tube section connected to each other, a diameter of the first tube section is greater than that of the second tube section, and both the vaporization core and the vent tube are sleeved in the first tube section, where
the core body has a run-through hole inside, the run-through hole is in communication with the interior of the second tube section, and the run-through hole and the second tube section are combined to form an airflow channel; and an air passing gap is reserved between a transition connection portion at which the second tube section is connected to the first tube section and the vaporization core, and the air passing gap is in communication with the airflow channel and the vent channel.

In an embodiment, a protruding stage is formed on an outer circumferential surface of the base body, and the vent tube is sleeved outside the base body and located between the transition connection portion and the protruding stage.

In an embodiment, one end of the vent tube in the axial direction is supported on the protruding stage, and a gap is reserved between the other end of the vent tube in the axial direction and the transition connection portion; and
a gap is reserved between the vent tube and each of the base body and the sleeve in the radial direction, or the vent tube is fixedly sleeved with the base body, and a gap is reserved between the vent tube and the sleeve in the radial direction.

In an embodiment, the vent tube is fixedly sleeved on the inner wall of the sleeve, and a gap is reserved between the vent tube and each of the outer circumferential surface of the base body and the protruding stage.

In an embodiment, the vent tube is a fiberglass tube.

An electronic vaporization device is provided, including the foregoing vaporizer.

Reference numerals: 100. vaporizer; 10. shell; 20. liquid storage cavity; 30. sleeve; 32. liquid inlet hole; 33. first tube section; 35. second tube section; 37. transition connection portion; 50. vaporization core; 52. base body; 521. through hole; 523. vent groove; 53. protruding stage; 54. core body; 70. vent tube; 80. vent channel.

To make the foregoing objects, features and advantages of the utility model more comprehensible, detailed description is made to specific implementations of the utility model below with reference to the accompanying drawings. In the following description, many specific details are described for thorough understanding of the utility model. However, the utility model can be implemented in many other manners different from those described herein. A person skilled in the art may make similar improvements without departing from the connotation of the utility model. Therefore, the utility model is not limited to the specific embodiments disclosed below.

In the description of the utility model, it should be understood that, orientation or position relationships indicated by terms such as “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, “axial”, “radial”, and “circumferential” are orientation or position relationship shown based on the accompanying drawings, and are merely used for describing the utility model and simplifying the description, rather than indicating or implying that the mentioned apparatus or element should have a particular orientation or be constructed and operated in a particular orientation, and therefore, should not be construed as a limitation to the utility model.

In addition, the terms “first” and “second” are used for descriptive purposes only and are not to be construed as indicating or implying relative importance or implicitly indicating the quantity of technical features indicated. Therefore, a feature restricted by “first” or “second” may explicitly indicate or implicitly include at least one of such features. In the description of the utility model, unless otherwise explicitly defined, “a plurality of” means at least two, for example, two, three, and the like.

In the utility model, unless otherwise expressly specified and limited, the terms such as “installation”, “link”, “connection” and “fixation” should be understood in a broad sense, for example, it may be fixed connection or detachable connection, or integration; and it may be mechanical connection or electrical connection; and it may be direct link or indirect link through an intermediary, and it may be internal communication of two elements or interaction between two elements, unless specifically limited otherwise. A person of ordinary skill in the art may understand the specific meanings of the foregoing terms in the utility model according to specific situations.

In the utility model, unless otherwise explicitly specified and defined, a first feature “on” or “below” a second feature may mean that the first feature and the second feature are in direct contact, or the first feature and the second feature are in indirect contact through an intermediary. In addition, that the first feature is “above”, “over”, or “on” the second feature may indicate that the first feature is directly above or obliquely above the second feature, or may merely indicate that a horizontal height of the first feature is higher than that of the second feature. That the first feature is “below”, “under”, and “beneath” the second feature may indicate that the first feature is directly below or obliquely below the second feature, or may merely indicate that the horizontal height of the first feature is lower than that of the second feature.

It should be noted that, when a component is referred to as “being fixed to” or “being arranged on” another component, the component may be directly on the another component, or there may be an intermediate component. When an element is considered to be “connected to” another element, the element may be directly connected to the another element, or an intermediate element may also be present. The terms “vertical”, “horizontal”, “upper”, “lower”, “left”, “right”, and similar expressions used in this specification are only for purposes of illustration but not indicate a unique implementation.

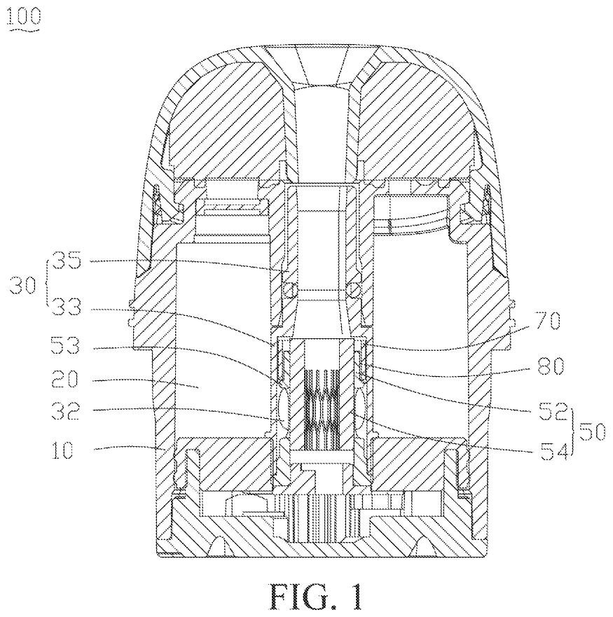

Referring to FIG. 1, in an embodiment of the utility model, a vaporizer 100 is provided, including a shell 10 and a vaporization assembly. The vaporization assembly includes a sleeve 30 and a vaporization core 50, where the sleeve 30 is arranged in the shell 10, a liquid storage cavity 20 is defined between the shell 10 and the sleeve 30, the vaporization core 50 is assembled in the sleeve 30, and a liquid inlet hole 32 communicating the vaporization core 50 with the liquid storage cavity 20 is provided on the sleeve 30. An aerosol-generation substrate in the liquid storage cavity 20 can directly flow to the vaporization core 50 through the liquid inlet hole 32, to supply liquid to the vaporization core 50.

In addition, the vaporizer 100 further includes a vent tube 70, where the vent tube 70 is sleeved between the sleeve 30 and the vaporization core 50, and spaced apart from at least one of the sleeve 30 or the vaporization core 50 to form a vent channel 80, where the vent channel 80 is capable of being in communication with the liquid inlet hole 32 and the outside. In this way, by arranging the vent tube 70 between the vaporization core 50 and the sleeve 30, a gap between the vent tube 70 and at least one of the vaporization core 50 or the sleeve 30 forms the vent channel 80. When liquid feeding of the aerosol-generation substrate is from the liquid storage cavity 20 and the liquid flows to the vaporization core 50, the external air can flow to the liquid inlet hole 32 and the liquid storage cavity 20 through the vent channel 80, to prevent formation of a negative pressure in the liquid storage cavity 20, and keep the air pressure in the liquid storage cavity 20 in balance with the atmospheric pressure outside, thereby ensuring smooth liquid feeding.

In some embodiments, the gap of the vent channel 80 ranges from 0.05 mm to 0.15 mm, and the relatively narrow gap of the vent channel 80 can form a capillary effect. Specifically, the vent channel 80 can have a ventilation state and a closed state. In the closed state, the air pressure in the liquid storage cavity 20 is in balance with the air pressure outside, and the inside of the vent channel 80 is sealed by a liquid film formed by the capillary effect. In other words, the vent tube 70 is arranged between the sleeve 30 and the vaporization core 50, and the space between the sleeve 30 and the vaporization core 50 is divided by the vent tube 70, so that both a gap between the vent tube 70 and the sleeve 30 and a gap between the vent tube 70 and the vaporization core 50 are very small, and then when the air pressure inside is in balance with the air pressure outside, the aerosol-generation substrate in the liquid storage cavity 20 can enter the vent channel 80 by using the capillary effect, and a liquid film blocking the vent channel 80 is formed, to close the vent channel 80 and prevent the aerosol-generation substrate in the liquid storage cavity 20 from leaking through the vent channel 80.

Moreover, when in the ventilation state, a negative pressure is generated in the liquid storage cavity 20, the liquid film ruptures under the action of the atmospheric pressure outside, and the vent channel 80 communicates the liquid inlet hole 32 with the outside, so that an external airflow can enter the liquid storage cavity 20 through the vent channel 80, to keep the air pressure in the liquid storage cavity 20 in balance with the atmospheric pressure outside during liquid feeding, thereby preventing obstructed liquid feeding.

In some embodiments, the vaporization core 50 includes a base body 52 and a core body 54, the base body 52 is sleeved in the sleeve 30, and the core body 54 is sleeved in the base body 52. A through hole 521 communicating the liquid inlet hole 32 with the core body 54 is provided on the base body 52, and the aerosol-generation substrate in the liquid storage cavity 20 passes through the liquid inlet hole 32 and then flows to the core body 54 through the through hole 521 on the base body 52, to directly supply liquid to the core body 54 in the base body 52. In addition, the vent tube 70 is sleeved between the base body 52 and the sleeve 30, and sleeved with at least one of the base body 52 or the sleeve 30 in a spaced-apart manner to form the vent channel 80, so that the gap between the vent tube 70 and at least one of the base body 52 or the sleeve 30 forms the vent channel 80 to ensure smooth liquid feeding; and when the vaporizer 100 is not in use, the liquid film in the vent channel 80 is used for sealing to prevent liquid leakage.

Further, the core body 54 is constructed as inner cotton wrapping heating wires, so that in the assembly process, it is only necessary to insert the inner cotton wrapping the heating wires into the base body 52, and then put the base body 52 into the sleeve 30. Outer cotton wound around the base body 52 in a conventional cotton core body 54 is eliminated to improve the convenience of assembly. In addition, the vent channel 80 is defined by the vent tube 70, to prevent obstructed liquid feeding.

In some embodiments, the sleeve 30 includes a first tube section 33 and a second tube section 35 connected to each other, a diameter of the first tube section 33 is greater than that of the second tube section 35, and both the vaporization core 50 and the vent tube 70 are sleeved in the first tube section 33. The core body 54 has a run-through hole inside, the run-through hole is in communication with the interior of the second tube section 35, and the run-through hole and the second tube section are combined to form an airflow channel. When flowing through the airflow channel, the external airflow can carry the aerosol-generation substrate emitted by the core body 54 to flow outward for the user to inhale. In addition, an air passing gap is reserved between a transition connection portion 37 at which the second tube section 35 is connected to the first tube section 33 and the vaporization core 50, and the air passing gap communicates the airflow channel with the vent channel 80, so that during liquid feeding, the external air can flow through the airflow channel to the vent channel 80 through the air passing gap, and finally flow to the liquid storage cavity 20, to balance the air pressure in the liquid storage cavity 20 with the air pressure outside, thereby ensuring smooth liquid feeding.

Further, a protruding stage 53 is formed on an outer circumferential surface of the base body 52, and the vent tube 70 is sleeved outside the base body 52 and located between the transition connection portion 37 and the protruding stage 53. In this way, the vent tube 70 can be limited by the protruding stage 53, to limit the vent tube 70 to a position close to the transition connection portion 37, thereby facilitating the communication between the vent channel 80 and the air passing gap at the transition connection portion 37.

In some embodiments, the vent tube 70 is fixedly sleeved on the base body 52, that is, the vent tube 70 fits the base body 52 with zero gap or the vent tube 70 perfectly fits the base body 52, and a gap is reserved between the vent tube 70 and the sleeve 30 in a radial direction and the vent channel 80 is defined. In this way, the vent tube 70 is fixedly assembled on the base body 52, and the gap between the vent tube 70 and the sleeve 30 in the radial direction is used to form the vent channel 80.

Specifically, one end of the vent tube 70 in the axial direction is supported on the protruding stage 53, and a gap is reserved between the other end of the vent tube 70 in the axial direction and the transition connection portion 37, so that the vent tube 70 is limitedly fixed on the protruding stage 53 of the base body 52, and a gap is reserved between the vent tube 70 and the transition connection portion 37 of the sleeve 30, which allows the airflow flowing in from the air passing gap to flow to the vent channel 80 between the vent tube 70 and the sleeve 30 after passing through the gap between the vent tube 70 and the transition connection portion 37, thereby ensuring smooth liquid feeding.

In some other embodiments, the vent tube 70 is fixedly sleeved on an inner wall of the sleeve 30, and a gap is reserved between the vent tube 70 and the base body 52 in a radial direction and the vent channel 80 is defined. In this way, the vent tube 70 is mounted and fixed through the sleeve 30, and the vent channel 80 is formed by using the gap between the vent tube 70 and the base body 52 in the radial direction, to ensure effective liquid feeding.

Specifically, a gap is reserved between the vent tube 70 and each of the outer circumferential surface of the base body 52 and the protruding stage 53, and the protruding stage 53 will not be completely in contact with the vent tube 70 to block the vent channel 80. In addition, the protruding stage 53 may be configured to reduce the diameter of the vent channel 80, to facilitate forming a capillary effect when no liquid is fed.

In some other embodiments, a gap is reserved between the vent tube 70 and each of the sleeve 30 and the base body 52 in a radial direction and the vent channel 80 is defined. In this way, vent channels 80 can be formed on both an inner side and an outer side of the vent tube 70, and the diameter of the vent channel 80 is relatively small, which is more convenient to use the capillary effect to form a liquid film, thereby achieving better sealing effects when no liquid is fed.

Specifically, one end of the vent tube 70 in the axial direction is supported on the protruding stage 53, and a gap is reserved between the other end of the vent tube 70 in the axial direction and the transition connection portion 37, so that when an external airflow flows to the vent tube 70, the external airflow is divided into two paths, where one path of external airflow flows into the vent channel 80 between the vent tube 70 and the sleeve 30, and the other path of external airflow flows into the vent channel 80 between the vent tube 70 and the base body 52. In addition, because a gap is reserved between the vent tube 70 and the transition connection portion 37, this path of airflow can still flow out between the protruding stage 53 and the vent tube 70, which is equivalent to that the vent tube 70 is movably supported on the protruding stage 53, and a flow space can be obtained through avoidance when the airflow flows through, thereby not affecting flowing of the airflow, and ensuring smooth liquid feeding.

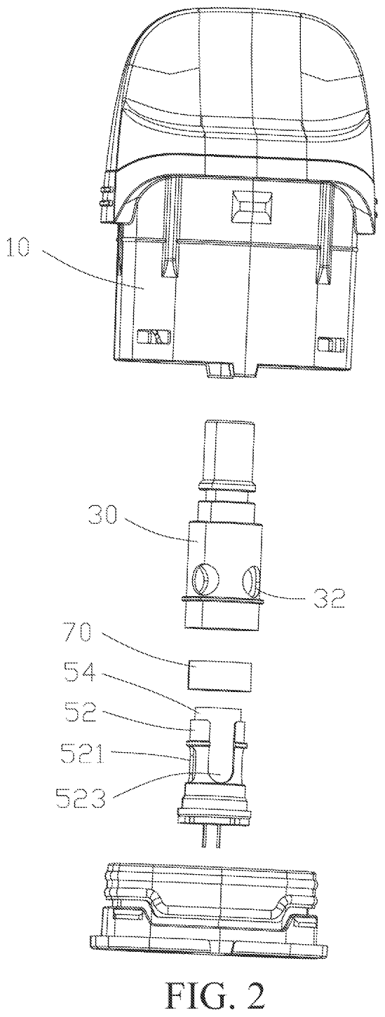

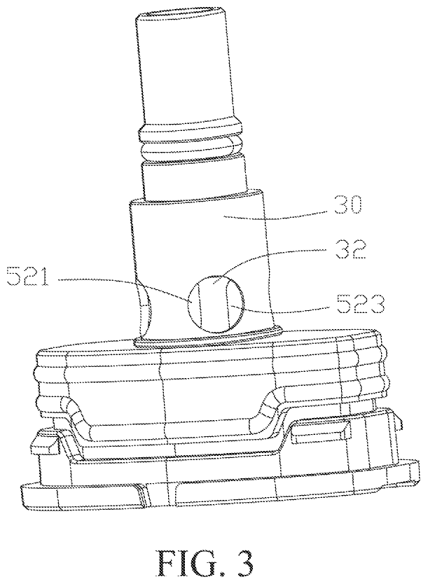

Referring to FIG. 2 and FIG. 3, in still some other embodiments, a vent groove 523 extending and provided in an axial direction of the vent tube 70 is provided on the base body 52, one part of the vent groove 523 is inside the vent tube 70, and the other part of the vent groove 523 extends to be in communication with the through hole 521, and the vent groove 523 is constructed to be the vent channel 80, to guide the external airflow to flow to the liquid inlet hole 32 through the through hole 521, thereby ensuring smooth liquid feeding. That is, a part of the base body 52 is removed, and when the vent tube 70 is sleeved outside the base body 52, the vent channel 80 is directly defined between the vent tube 70 and the core body 54 inside the base body 52.

Specifically, one end of the vent groove 523 away from the through hole 521 is open, which allows the external airflow to flow into the vent tube 70 and then directly enter the vent groove 523, thereby ensuring that the external airflow flows to the vent groove 523 smoothly.

Further, the vent tube 70 is sleeved with the base body 52 with zero gap or the vent tube 70 is in interference fit with the base body 52, and the vent tube 70 is sleeved with the sleeve 30 with zero gap or the vent tube 70 is in interference fit with the sleeve 30. The manner of mounting the vent tube 70, the base body 52, and the sleeve 30 is not limited herein.

In any one of the foregoing embodiments, the vent tube 70 is a fiberglass tube, so that it is very convenient to remove the outside wrapping process and use a fiberglass tube to assemble the vaporization assembly. Moreover, the fiberglass tube can make the consistency of ventilation more stable. In addition, the aerosol-generation substrate in the liquid storage cavity 20 can be directly in contact with the vaporization core 50 through the liquid inlet hole 32 on the sleeve 30, thereby improving the liquid guiding speed and stability. Further, optionally, the shell 10 has a suction nozzle, and the vent tube 70 is located at one end of the sleeve 30 close to the suction nozzle, to form a vent channel 80 between the suction nozzle and the liquid inlet hole 32, which allows the external airflow to enter the vent channel 80 through the suction nozzle, thereby ensuring smoothness of liquid feeding.

Based on the same concept, in an embodiment of the utility model, an electronic vaporization device is provided, including the foregoing vaporizer 100. The vaporizer 100 includes a shell 10, a sleeve 30, and a vaporization core 50, where the sleeve 30 is arranged in the shell 10, a liquid storage cavity 20 is defined between the shell 10 and the sleeve 30, the vaporization core 50 is assembled in the sleeve 30, and a liquid inlet hole 32 communicating the vaporization core 50 with the liquid storage cavity 20 is provided on the sleeve 30. An aerosol-generation substrate in the liquid storage cavity 20 can directly flow to the vaporization core 50 through the liquid inlet hole 32, to supply liquid to the vaporization core 50.

In addition, the vaporizer 100 further includes a vent tube 70, where the vent tube 70 is sleeved between the sleeve 30 and the vaporization core 50, and spaced apart from at least one of the sleeve 30 or the vaporization core 50 to form a vent channel 80, where the vent channel 80 is capable of being in communication with the liquid inlet hole 32 and the outside. In this way, by arranging the vent tube 70 between the vaporization core 50 and the sleeve 30, a gap between the vent tube 70 and at least one of the vaporization core 50 or the sleeve 30 forms the vent channel 80. When liquid feeding of the aerosol-generation substrate is from the liquid storage cavity 20 and the liquid flows to the vaporization core 50, the external air can flow to the liquid inlet hole 32 and the liquid storage cavity 20 through the vent channel 80, to prevent formation of a negative pressure in the liquid storage cavity 20, and keep the air pressure in the liquid storage cavity 20 in balance with the atmospheric pressure outside, thereby ensuring smooth liquid feeding.

Various technical features in the foregoing embodiments may be combined randomly. For a concise description, possible combinations of various technical features in the foregoing embodiments are not all described. However, the combinations of the technical features are to be considered as falling within the scope recorded in this specification provided that the combinations of the technical features do not conflict with each other.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A vaporizer, comprising:
a shell;
a vaporization assembly comprising a sleeve and a vaporization core, the sleeve being arranged in the shell, a liquid storage cavity being defined between the shell and the sleeve, the vaporization core being arranged in the sleeve, and a liquid inlet hole communicating the vaporization core with the liquid storage cavity being provided on the sleeve; and
a vent tube sleeved between the sleeve and the vaporization core, and spaced apart from at least one of the sleeve or the vaporization core to form a vent channel,
wherein the vent channel is configured to be in communication with the liquid inlet hole and an outside.

2. The vaporizer of claim 1, wherein a gap of the vent channel ranges from 0.05 mm to 0.15 mm.

3. The vaporizer of claim 1, wherein the vaporization core comprises a base body and a core body, the base body being arranged in the sleeve, and the core body being at least partially mounted on the base body,
wherein a through hole communicating the liquid inlet hole with the core body is provided on the base body, and
wherein the vent tube is sleeved between the base body and the sleeve, and sleeved with at least one of the base body or the sleeve in a spaced-apart manner to form the vent channel.

4. The vaporizer of claim 3, wherein the vent tube is fixedly sleeved on the base body, and a gap is reserved between the vent tube and the sleeve in a radial direction so as to define the vent channel.

5. The vaporizer of claim 3, wherein the vent tube is fixedly sleeved on an inner wall of the sleeve, and a gap is reserved between the vent tube and the base body in a radial direction so as to define the vent channel.

6. The vaporizer of claim 3, wherein a gap is reserved between the vent tube and each of the sleeve and the base body in a radial direction so as to define the vent channel.

7. The vaporizer of claim 3, wherein a vent groove extending and provided in an axial direction of the vent tube is provided on the base body, one part of the vent groove being inside the vent tube, and an other part of the vent groove extending to be in communication with the through hole, and
wherein the vent groove comprises the vent channel.

8. The vaporizer of claim 4, wherein the sleeve comprises a first tube section and a second tube section connected to each other, a diameter of the first tube section being greater than a diameter of the second tube section, and both the vaporization core and the vent tube are sleeved in the first tube section,
wherein the core body has a run-through hole inside, the run-through hole being in communication with and interior of the second tube section, and the run-through hole and the second tube section being combined to form an airflow channel, and
wherein an air passing gap is reserved between a transition connection portion at which the second tube section is connected to the first tube section and the vaporization core, the air passing gap being in communication with the airflow channel and the vent channel.

9. The vaporizer of claim 8, wherein a protruding stage is formed on an outer circumferential surface of the base body, and the vent tube is sleeved outside the base body and located between the transition connection portion and the protruding stage.

10. The vaporizer of claim 9, wherein one end of the vent tube in the axial direction is supported on the protruding stage, and a gap is reserved between an other end of the vent tube in the axial direction and the transition connection portion, and a gap is reserved between the vent tube and each of the base body and the sleeve in the radial direction, or
wherein the vent tube is fixedly sleeved with the base body, and a gap is reserved between the vent tube and the sleeve in the radial direction.

11. The vaporizer of claim 9, wherein the vent tube is fixedly sleeved on an inner wall of the sleeve, and a gap is reserved between the vent tube and each of an outer circumferential surface of the base body and the protruding stage.

12. The vaporizer of claim 1, wherein the vent tube comprises a fiberglass tube.

13. An electronic vaporization device, comprising:
the vaporizer of claim 1.
